(19) United States
(12) Patent Application Publication
(10) Pub. No.: US 2025/0111942 A1
(43) Pub. Date: Apr. 3, 2025

(54) SYSTEM AND METHOD FOR THE DIAGNOSIS OF VULVOVAGINAL CONDITIONS

(71) Applicant: MOR RESEARCH APPLICATIONS LTD., Ramat Gan (IL)

(72) Inventors: Arnon AGMON, Givaataym (IL); Ran KEREN, Tel Aviv (IL)

(21) Appl. No.: 18/832,919

(22) PCT Filed: Jan. 26, 2023
(86) PCT No.: PCT/IB2023/050656

§ 371 Date: Jul. 24, 2024

(30) Foreign Application Priority Data
Jan. 26, 2022 (SG) 10202200810X

(51) Int. Cl. G16H 50/20; G16H 10/60

(52) U.S. Cl. CPC G16H 50/20; G16H 10/60

(57) ABSTRACT

The present disclosure relates to a system and methods for remote medical diagnosis and therapy and more particularly, for the patient driven diagnosis of vaginitis using multi sourced data.

### CROSS-REFERENCE TO RELATED APPLICATIONS

The present application claims the benefit of Singapore Provisional Application No. 10202200810X, filed on 26 Jan. 2022, the disclosure of which is hereby incorporated in its entirety by reference herein.

### FIELD OF THE DISCLOSURE

The present disclosure relates to a system and method for remote medical diagnosis and therapy and more particularly, for the patient driven diagnosis of vaginitis using multi sourced data.

### BACKGROUND

The most common reason for Gynecological visits by adult women is due to various forms of vulvovaginal infections. The most common vaginal infections include bacterial vaginosis (BV), candida infection and trichomonas infection. The most common reason for vulvar complaints is contact dermatitis. It is estimated that about half of all professional diagnosis of vaginitis is erroneous. Vulvovaginal conditions are commonly associated with very disturbing symptoms, which makes this condition an urgent matter for the sufferer. The increasing low availability of physicians and long wait time for appointment induce self-diagnosis and self-medication, which in most instances is not curative and lowers diagnosis accuracy by physician.

It is estimated that about half of all professional diagnosis of Vaginitis is erroneous. This high diagnosis failure rate is likely due to overlapping signs and symptoms of the various vaginal conditions. Other factors affecting diagnostic accuracy include a relatively long diagnostic procedure, around 20 minutes, a need for multiple tests and tools required for diagnosis (pH meter, KOH test, microscopy, culture, immunoassays, DNA tests and so forth) as well as medical expertise, which most practicing gynecologists do not possess. This high diagnostic failure rate leads to incorrect therapy, worsening of the condition, increased suffering and repeat visits. The arduous diagnostic process is also in the heart of a vicious cycle of low diagnostic performance leading to therapy failure leading to repeat visits, high cost for insurer and increased patient suffering.

Telemedicine has become very common in recent years, and especially during and following the Coronavirus (COVID-19) pandemic. Both patients and physicians accept the use of telemedicine as a part of providing and receiving health services. Many Health Maintenance Organizations (HMOs) are now providing financial incentives for physicians to engage with patients using telemedicine. This saves time and money for all parties involved. With the aim of providing more services via telemedicine, especially those requiring diagnosis of medical conditions, various accessory devices are already in the market today. However, correct and reliable diagnosis relies on a combination of subjective complaints, visual inspection, physical signs, and microscopic findings. Laboratory techniques to detect culprit organisms such as culture, DNA and antigen detection tests are now clinically accepted as an alternative to physical and microscopic findings.

The lengthy procedure of vaginitis diagnosis, the need for complex instrument and high proficiency combined with rising costs of healthcare remains the major hurdles for providing reliable vaginitis diagnosis accessible for a wider community.

In view of the foregoing, therefore there is a need to provide a method and a system that overcome or at least ameliorate the above limitations.

### SUMMARY

There is provided a method for diagnosing a vaginal disorder in a subject. As can be appreciated, the subject is a female individual. The method may comprise collecting primary data obtained from a device for diagnosing the vaginal disorder, storing the primary data in a database, sending the primary data to a processor, wherein the processor is configured to run a plurality of modules comprising at least a diagnosis module and a treatment module and to process the primary data and determining the likelihood of the subject diagnosed with the vaginal disorder based on the data processed and if the subject is diagnosed with the vaginal disorder, recommending one or more treatment options.

Advantageously, the method provided in the present disclosure may provide accurate diagnosis and subsequently recommend a suitable treatment option to the subject, when necessary.

Further advantageously, since the process may be undertaken remotely without (or with minimal) assistance from a healthcare professional, the cost for the diagnosis may be minimized.

In accordance with some embodiments of the present disclosure, the method may further comprise collecting secondary data. Optionally, said secondary data may comprise structured data and non-structured data. The secondary data may be fed to the processor for processing.

The diagnosis of the vaginal disorder may comprise diagnosing candidiasis, bacterial vaginosis and trichomoniasis or vaginitis. The diagnosis of the vaginal disorder may further comprise diagnosing vulvar conditions. The diagnosis of vulvar conditions may comprise diagnosis of vulvar contact dermatitis. Optionally, the processor may further comprise a computing element and a machine learning module, wherein said computing element may comprise a diagnosis module and a treatment module. Optionally, the determination of the likelihood of the subject diagnosed with the vaginal disorder may be based on a probability score generated by the computing element.

In accordance with some embodiments of the present disclosure, the primary data may comprise at least one of the followings: pH, result of candida antigen test, result of sialidase activity test, result of DNA or RNA test, one or more images obtained from the subject. Optionally, the primary data may comprise the combination of the aforementioned test results. Optionally, the diagnosis may further comprise collecting secondary data. The secondary data may comprise asking the subject if the subject is experiencing at least one of the following conditions: external vulvar pain, itching sensation and external vulvar discomfort.

Optionally, during the diagnosis process, if the sialidase activity test is positive, the subject may be asked if the subject is experiencing at least one of the following conditions: fishy smell and increased or abnormal discharge. Optionally, during the diagnosis, if the pH is greater than 4.5, the subject may be recommended to undergo a trichomonas antigen test. If the result of the antigen test is negative, the subject may be asked if the subject is experiencing at least one of the following conditions: fishy smell and increased or abnormal discharge.

If the result of the antigen test is negative, during the diagnosis process, the one or more images may be analyzed by an image analysis module. Optionally, if the result of the candida antigen test is negative and the pH is less than 4.5, the one or more images may be analyzed by the image analysis module to confirm the presence of Candida organism.

There is provided a system for diagnosing a vaginal disorder in a subject. The system may comprise a device for analyzing a sample obtained from the subject, a database for storing primary data, said primary data comprising a test result from said device and a processor, configured to process the primary data and to run a plurality of modules comprising at least a diagnosis module and a treatment module. The system is useful for providing the likelihood of the vaginal disorder in the subject based on the data processed.

Optionally, the device for analyzing the sample may comprise a collection apparatus for collecting and sampling vaginal discharge or fluid and a sensor for processing the sampling vaginal discharge or fluid, said sensor comprising at least two lateral flow assays (LFAs). Optionally, the system may further comprise an amine sensor to detect the presence of one or more amine containing compounds.

In accordance with some embodiments of the present disclosure, the database of the system may further comprise secondary data. The secondary data may comprise structured data and non-structured data. Optionally, the system may be suitable for diagnosing candidiasis, bacterial vaginosis and trichomoniasis or vaginitis.

The processor of the system may further comprise a computing element and a machine learning module, wherein said computing element may comprise a diagnosis module and a treatment module. In accordance with some embodiments of the present disclosure, the likelihood of the subject diagnosed with the vaginal disorder may be based on a probability score generated by the computing element of the system. The primary data stored in the database may comprise at least one of the followings: pH, result of candida antigen test, result of sialidase activity test, result of DNA or RNA test, one or more images obtained from the subject. The primary data may comprise the combination of the aforementioned test results.

Advantageously, the method and system described herein, in addition to providing diagnosis to the subject, may provide recommendations including treatment options and refer the subject to medical consultation.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of a system and method for diagnosis of vulvo-vaginitis, according to some embodiment of the disclosure;

FIG. 2 is a schematic illustration of a method for diagnosis of candidiasis, according to some embodiment of the disclosure;

FIG. 3 is a schematic illustration of a method for diagnosis of bacterial vaginosis and/or Trichomoniasis, according to some embodiment of the disclosure;

FIG. 4. is a schematic illustration of a method for diagnosis of vaginitis, according to some embodiment of the disclosure; and

FIG. 5 is a table showing an example of objective and subjective parameters probabilities calculations used in conjunction with the method described in relation to FIG. 4, according to an embodiment of the disclosure.

### DETAILED DESCRIPTION

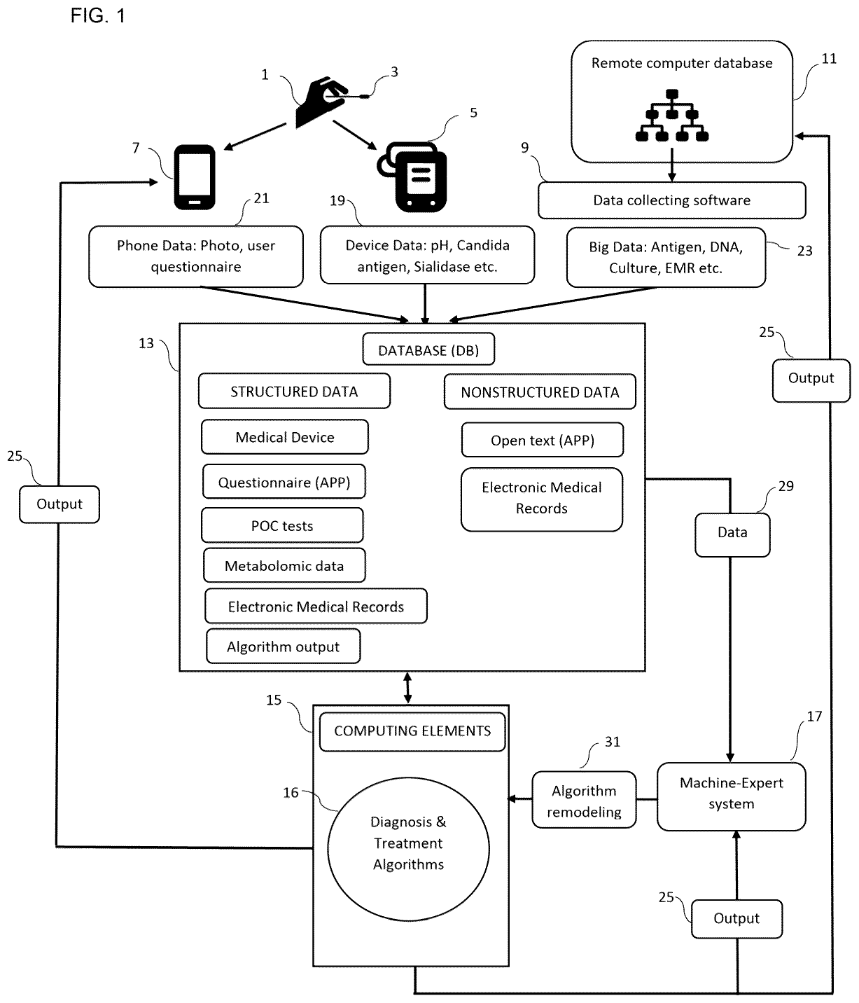

FIG. 1 illustrates a system described in the present disclosure. According to some embodiments, the system disclosed herein includes user 1 with sample 3, a device 5 and a mobile phone 7. In some embodiments, user 1 is a female person or female individual with preferably one or more vaginal symptoms provoking her to seek medical attention. Sample 3 is preferably a vaginal sample of vaginal discharge. In some embodiments, sample 3 may comprise one or more samples obtained from one or more sampling attempts. In some embodiments, sample 3 may be obtained from a female individual suspected to have a vaginal disorder. The sample may be provided using a swab including a specialized swab or pad. Device 5 may include a medical device such as previously described in Singapore patent application number 10202109369P or in the international application number application number PCT/IB2022/057958, which is hereby incorporated by reference in its entirety. In some embodiments, said device or medical device 5 is used for analyzing vaginal samples using various objective tests. In some embodiments, medical device 5 is for analyzing sample 3 obtained from the subject 1 who is a female individual. In some embodiments, medical device 5 may include but not limited to pH meter, antigenic test or DNA test and also image acquiring device used in identifying microorganisms culpable in vaginitis and the like. In some embodiments, other suitable communication devices other than mobile phone or smartphone 7 having the same functionality may be used in lieu of mobile phone 7. This includes a tablet (including iPad), a smart watch, a smart garment and the like. It should be evident that many users, samples, medical devices, and phones may be used to collect data. In some embodiments, the system described in the present disclosure may also include a data collecting software 9 designed to collect data from various remote computers databases 11 including but not limited to database from health maintenance organizations (HMO's) and points of care such as clinics and hospitals.

In some embodiments, the system described in the present disclosure may further include one or more databases (DB) 13 for storing data. In some embodiments, the data may be primary data and/or secondary data as will be described below. In some embodiments, the system may further include a computing element 15 and optionally a machine-expert system 17. In some embodiments, the one or more databases (DB) 13 may use Oracle™, SQL™, Accumolo™ or a similar software. In some embodiments, computing element 15 may include one or more central processing units (CPUs), graphical processing units (GPU) as well as other computing elements. These computing elements are used for processing the one or more databases (DB) 13 data as well as diagnostic and treatment module 16 also described in later figures. In some embodiments, the processor mentioned above including the CPUs, is configured to process the data (including primary and/or secondary data). In some embodiments, the processor is also configured to run a plurality of modules. The plurality of modules comprises at least a diagnosis module and a treatment module, for providing the likelihood of the vulvo-vaginal disorder in the subject based on the data processed. In some embodiments, the plurality of modules optionally comprises an image or photo analysis module.

In some embodiments, primary data may include pH, result of candida antigen test, result of sialidase activity test, result of DNA or RNA test, one or more images obtained from the subject and combination thereof. In some embodiments, the primary data may include answers to the questions in a questionnaire. Said questions may include: asking if the subject is experiencing at least one of the following conditions: external vulvar pain, itching sensation and external vulvar discomfort.

In some embodiments, each of the primary data and the secondary data may include structured data and non-structured (or unstructured) data. In some embodiments, the structured data may include results obtained from the medical device such as device 5, questionnaire, results obtained from point of care tests, metabolomic data (including those obtained from Raman spectrometer), electronic medical report and module output, including algorithm output (see FIG. 1). In some embodiments, the non-structured data may include open text and electronic medical report.

In some embodiments, the one or more databases 13 is configured to store data including test results from medical device 5. As an illustrative example, the pH readings, result of antigen tests and result of DNA/RNA test may be stored in database 13.

In some embodiments, medical device 5 may comprise a collection apparatus for collecting and sampling vaginal discharge, vulvar sample or fluid and a sensor for processing the sampling vulvo-vaginal discharge or fluid, said sensor comprising at least two lateral flow assays (LFAs).

According to some embodiments of the disclosure, the method for diagnosing a vaginal disorder may include user 1 taking self-sample 3 from a pad, a vulvar sample, or a vaginal discharge. It is to be appreciated that the sample 3 can also be taken by a medical professional and used in a similar fashion. In some embodiments, sampler can be a pad, a simple cotton headed stick, a simple brush or other specialized sampler such as device described Singapore patent application number 10202109369P or in the international application number PCT/IB2022/057958. In some embodiments, the sample or sampler may be photographed, and laser imaging, detection and ranging (LIDAR) scanned using smartphone 7. In some embodiments, the smartphone 7 can be a smartphone using 3MP micro lens camera with X60 magnification, for example but not limited to Oppo Find X3 Pro phone from Guangdong Oppo Mobile Telecommunications Ltd, Guangdong, China.

According to some embodiments of the disclosure, sample 3 is then introduced to medical device 5 that analyzes said sample. Said analysis may be performed at least by one of the followings: pH readings, antigenic tests for candida species and trichomonas as well as Sialidase enzymatic reaction. Other tests including rapid DNA/RNA tests, other antigenic tests and the like may also be performed. In some embodiments, the analysis performed by device 5 includes pH readings, antigenic tests for candida species and trichomonas, and Sialidase enzymatic reaction. In some embodiments, the analysis performed by device 5 includes pH readings, antigenic tests for candida species and trichomonas, Sialidase enzymatic reaction and DNA/RNA tests. As is described herein, in some embodiments, the diagnosis of the vaginal disorder may further comprise diagnosing vulvar conditions. The diagnosis of vulvar conditions may comprise diagnosing vulvar contact dermatitis.

In some embodiments, the analysis performed by device 5 includes DNA/RNA tests and photo image acquisition and analysis.

In some embodiments, device 5 may further include additional sensor. For example, the additional sensor is suitable for detecting the presence of certain substance including base for example amine. It is acknowledged that the female person suspected to have bacterial vaginosis has fishy odor in the vaginal discharge. The fishy odor is due to the presence of amine containing substance including triethylamine. Hence, in some embodiments, device 5 may further include amine sensor. It is to be appreciated that the suitable amine sensor may be able to detect the presence of any amine containing compound or substance and is not limited to detecting simple amine such as triethylamine but more complex amines including aromatic compounds having amine as either one of the side chains of the aromatic compounds or amine present in the aromatic ring of the aromatic compounds.

In some embodiments, the additional sensor including amine sensor may be provided separately from device 5. In some embodiments, as described above, the additional sensor may be provided as part of the device and thus may be integrated with device 5.

In some embodiments, in bacterial vaginitis and trichomoniasis, vaginal pH is higher than 4.5 (for example when the pH is 5 or higher). In some embodiments, the presence of Trichomonas vaginalis in trichomoniasis is essential for the diagnosis. In some embodiments, the presence of candida species, for example Candida albicans or other Candida species or yeast in candidiasis is essential for diagnosis. In bacterial vaginosis (BV), vaginal pH is typically higher than 4.5 and sialidase enzyme activity has been shown to be specific and sensitive for the presence of BV. According to some embodiments of the disclosure, samples can also be analyzed for metabolomics data using a Raman spectrometer such as described in the device disclosed in Singapore patent application number 10202109369P or in the international application number PCT/IB2022/057958.

In some embodiments, the subject receives and answers a structured questionnaire. In some embodiments, the subject received and responded to the questionnaire using mobile phone or smartphone 7. The subject may optionally input unstructured (or non-structured) data items related to its condition. The non-structured data as can be seen from FIG. 1 may include open text and electronic medical records. This can be performed by smartphone 7 or other suitable user input devices. In some embodiments, device data 19 of sample 3 analysis and phone data 21 of sample 3 photo, LIDAR scan, structured and non-structured data may be transferred to database (DB) 13 and stored. Said user 1 data is then used by computing elements 15 to run diagnosis and treatment module 16 to provide output 25 comprising diagnosis and treatment options. Output 25 is then transferred to user 1 smartphone 7 for display. Optionally, output 25 may be stored in database (DB) 13, and transferred, together with other user data to health provider on remote computer database 11.

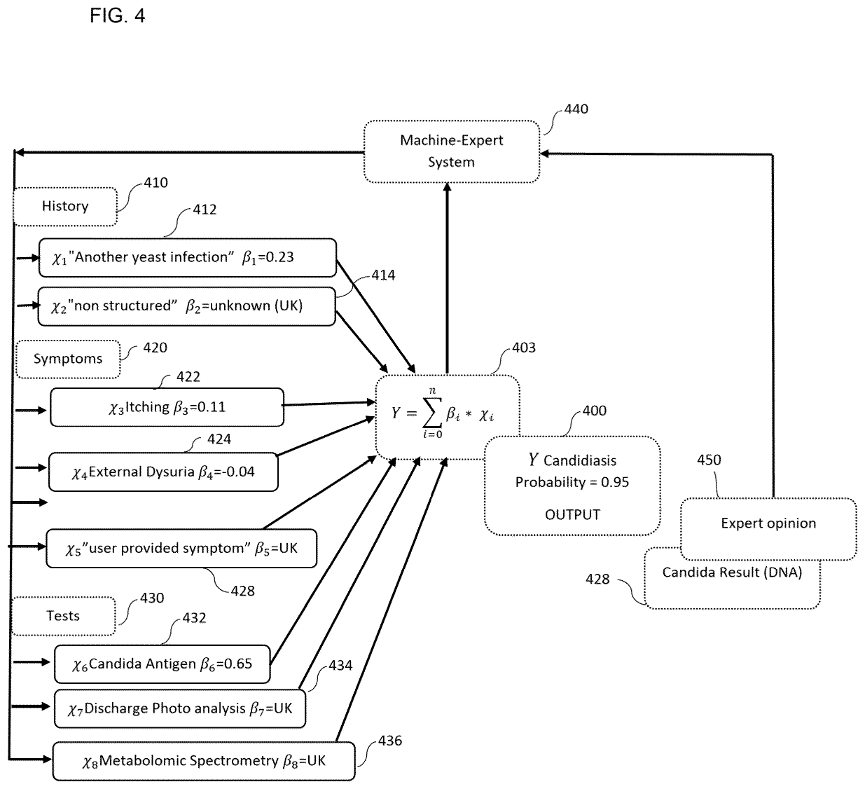

The method may further comprise sending output 25 and data 29 to machine-expert system 17 that is operative in remodeling module 31. An exemplary illustration is shown in FIG. 4 using structured data. However, it should be evident to the person skilled in the art that non-structured data from user 1 or remote data sources 11 may also be used to refine and enhance the methods disclosed herein. Accordingly, it is to be appreciated that both structured and non-structured data may be used to refine and enhance the methods described in the present disclosure.

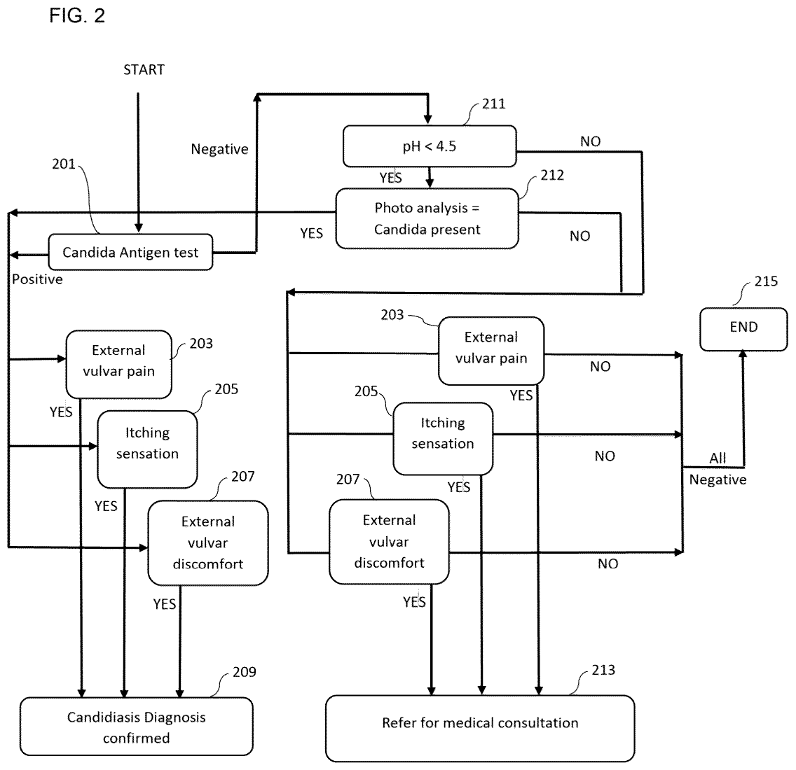

FIG. 2 describes a method for diagnosis of candidiasis, according to some embodiments of the disclosure. In some embodiments, the diagnosis of candidiasis requires the presence of candida organism (for example Candida albicans) in the vulvovaginal sample and at least one symptom that is bothersome to the patient and is related to candidiasis. In some embodiments, the presence of candida is insufficient since many women have a non-parasitic relationship with said organism. In some embodiments, candida antigen test may be highly sensitive and specific for the presence of candida species. The method described herein may advantageously overcome this drawback.

According to some embodiments of the disclosure, the method comprises identifying the results of candida antigen test, obtained from device 5 of FIG. 1. In some embodiments, if candida antigen test result 201 is positive, user 1 is prompted with the following questions through an application on her smartphone 7 or on device 5: Do you experience any of these conditions? “External vulvar pain” 203, “Itching sensation” 205, “External vulvar discomfort” 207. If one or more of the above is true, then diagnosis of candidiasis is confirmed 209. In some embodiments, if candida antigen test result 201 is negative, the system will check if the pH is less than 4.5 (211). In some embodiments, if the pH is less than 4.5, the system will request photo analysis 212 result of vulvovaginal sample 3 in FIG. 1.

In some embodiments, if photo analysis 212 indicates the presence of candida, the subject will be prompted the following questions: Do you experience any of these conditions? “External vulvar pain” 203, “Itching sensation” 205, “external vulvar discomfort” 207. If one or more of the above is true, then diagnosis of candidiasis is confirmed 209. In some embodiments, candida antigenic test 201 may occasionally miss the presence of candida. Advantageously, the method described herein may include using at least one of the followings: visual photo analysis, pH readings and subjective symptoms in addition to the antigen test. In some embodiments, photo analysis may be replaced by Raman spectrometer that is connected to (or in communication with) device 5. In some embodiments, the photo analysis may be used in conjunction with the analysis using Raman spectrometer. In some embodiments, the Raman spectrometer may be a mobile or a stationary Raman spectrometer. The metabolomic profile of candidiasis can then be elucidated by comparing readings from Raman spectrometer and data obtained from published data or data collected by the system described herein. In some embodiments, the diagnostic loop using photo analysis and/or Raman spectrometer may be implemented when candida photo analysis or candidiasis metabolomic snapshot reaches on par performance with candida antigenic tests when compared with the DNA tests.

In some embodiments, diagnosis with photo analysis and/or pH test is advantageously lower in cost compared with antigenic or DNA testing. In some embodiments, if pH readings 211 or photo analysis 212 fails to ensure diagnosis of candida, the user is prompted with the same questions; Do you experience any of these conditions? “External vulvar pain” 203, “Itching sensation” 205, “external vulvar discomfort” 207. If any of the above is true, the system disclosed herein will then prompt the user to refer for medical consultation 213. Advantageously, this step is to ensure that the user is not suffering from a condition with symptoms akin to candidiasis that is brought about by other factors. If the response for all three questions above is negative, the method ends 215 with no action performed by the system.

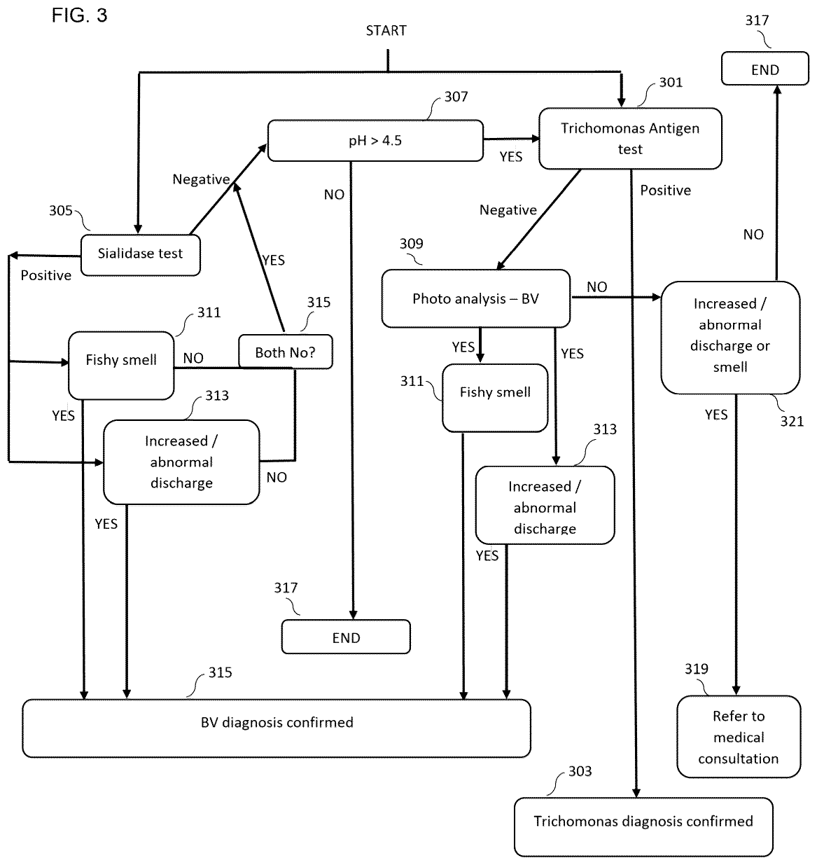

FIG. 3 illustrates a method for diagnosis of bacterial vaginosis (BV) and trichomoniasis, according to some embodiment of the disclosure. In some embodiments of the present disclosure, the method comprises identifying the results of trichomonas antigen test 301 as being positive or negative. In some embodiments, if trichomonas test is positive, trichomonas diagnosis is confirmed 303. In some embodiments, the presence of Trichomonas vaginalis in the vagina, in trichomoniasis, can never be a carrier state but is considered as a disease even if symptoms and signs are absent. The method may comprise identifying sialidase activity test results 305 as being negative or positive. In some embodiments, sialidase activity as discussed above is highly specific and sensitive for BV when compared with Nugent score.

In some embodiments, if sialidase activity test result 305 is positive, the user will be asked with the following questions: “Do you experience bad (or fishy) smell (rotten fish)” 311 and “Do you experience increased or abnormal discharge?” 313 and the method optionally comprises waiting for user's response. If the response to either one of the two questions is “YES”, then BV diagnosis 315 is confirmed. In some embodiments, if both answers 311 & 313 are “NO” (Both NO 315), then the system continues as if sialidase test 305 is negative. In this manner, the method advantageously may circumvent diagnosis and treatment relying solely on the result of sialidase activity test.

In some embodiments, if the sialidase test 305 is negative, the method prompts the subject whether pH readings are greater than 4.5 (307), for example pH 5 or higher. In some embodiments, if the pH reading 307 is less than or equal to 4.5, the method ends 317. In some embodiments, if the pH reading 307 is greater than 4.5, the user is asked if trichomonas antigen test 301 is positive. If the trichomonas antigen test 301 is positive, the trichomonas diagnosis is confirmed. In some embodiments, if the trichomonas antigen test 301 is negative, the system queries the result of discharge photo analysis 309. If the result of discharge photo analysis 309 is positive for BV, the system provides the user with the following questions: “Do you experience bad (or fishy) smell (rotten fish)” 311 and “Do you experience increased or abnormal discharge?” 313. In some embodiments, the method may optionally comprise waiting for user's response. In some embodiments, if the response to either one of the above questions is “YES”, then BV diagnosis 315 is confirmed. In some embodiments, if the result of the discharge photo analysis 309 is negative but the response to both questions 311, 313 is “YES”, the system will recommend the user to undergo a medical consultation 319.

The above additional step is beneficial as it is considered as a safety step implemented when the signs of vaginitis are identified but the system is unable to correlate it with clinical context. In some embodiments, if photo analysis 309 is not diagnostic of BV, the patient is prompted with same clinical questions 311, 313 (combined in square 321). In some embodiments, if all the questions are negated, then the method ends 317. In some embodiments, pH readings greater than 4.5 may be normal in some women and on its own does not require further investigation. If any of questions in square 321 is positive, the user is recommended or referred to medical consultation 319 for further investigation. It is to be understood by a person skilled in the art that other questions can be added to or can replace questions 311 and 313 following machine-expert learning process.

FIG. 4 describes a method for diagnosis of vaginitis in accordance with some embodiments of the disclosure. In some embodiments, the method for the diagnosis of vaginitis generates a probability score. The total probability of vaginitis is the summation of different factors, contributing to or suppressing the total probability of vaginitis.

In some embodiments, the method is described in the general Form (equation (i)):

$$Y = {\sum }_{i = 0}^{n} {β}_{i} * {χ}_{i} \qquad ( i )$$

where Y denotes the probability that a condition exists. In some embodiments, these conditions may include candidiasis, bacterial vaginosis and the like. X denote objective or subjective parameters. In some embodiments, objective parameters can include test such as culture, rapid antigen test, DNA analysis of vaginal or vulvar organisms, visual analysis of discharge, metabolomic profile using Raman spectrometry of discharge, electronic nose (e-nose) analysis of gases and the like. In some embodiments, the objective parameters may include history of current problem, habits such as vaginal douching, symptoms such as itch, description of bodily or mental state such as “feeling of swelling”, “feeling external dysuria” or “this is a recurring event” and the like.

In some embodiments, parameters X effecting the probability a condition is present, which take the value of 1, and β is the probability factor or effector (ranging from 0 to 1). In some embodiments, parameters X take the value of 1 or 0 (present or no data), while probability factor β takes the values of ±1 (+1 being present, −1 being absent, and these are mutually exclusive). In some embodiments, probability factor β takes the value of |0| for nonspecific (NS) results. Each of the above is used in the final formula. Initially, probabilities are calculated from likelihood ratios calculated from likelihood ratios (LR) using an approximation. Pre-test prevalence is assumed to lie between 10% and 90%. Pre-test prevalence if target conditions are in the range of 15%-50%. Additionally, cumulative post-test probabilities are added up to 100% or 0%. These initial estimates are accurate within 10% for all pre-test probabilities with an average error of about 4%. Subsequently, artificial intelligence (AI) operation will refine probabilities to reduce errors. In some embodiments, the change in probability (DP) is given by the following formula (equation (ii)):

$$Δ P = 0.19 * {log}_{e} LR \qquad ( ii )$$

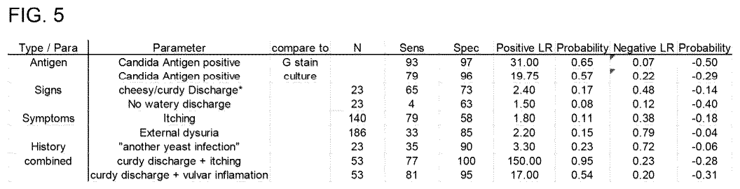

The table shown in FIG. 5 illustrates an example of objective and subjective parameters probabilities calculations. The same table can be implemented for all conditions calculated by the method described herein.

According to some embodiments of the disclosure and by referring to FIG. 4, the method proceeds when candidiasis probability 400 (Y) or output is calculated with summation module 403 using multitude of input parameters (Xᵢ). For clarity, in this exemplary embodiment, input parameters are sub divided into input categories such as history 410, symptoms 420 and tests 430. Other categories including habits, demographics, and the like may also be used and added into the method. Input parameters can be structured such as input candida antigen 432 and non-structured data such as user provided symptom 428. Input parameters can have predefined, unknown, or calculated probabilities (βᵢ). In some embodiments, pre calculated probabilities are calculated using existing literature data as described above. An example includes “another yeast infection” 412 β₁=0.23. In some embodiments, unknown probabilities are input parameters that have no pre-defined known probability on the outcome of candidiasis. In some embodiments, calculated probabilities are probabilities pre-defined or unknown probabilities that have been modified or calculated by the system and are now attached to specific parameter.

In this exemplary embodiment, the system starts by sending pre-determined parameters “Another yeast infection” 412 β₁=0.23, Itching 422 β₃=0.11, External dysuria 424 β₄=−0.04 and candida antigen 432 β₆=0.65. It should be noted that External dysuria 424 may have a negative probability score since pre-determined data showed that lack of such symptoms has a negative effect on the probability of candidiasis 400. Summation Module 403 determines candidiasis probability as shown in equation (iii):

$$Y = {\sum }_{i = 0}^{n} ( {0.23}_{1} * Another yeast {infection}_{1} ) + ( {0.11}_{3} * {Itching}_{3} ) + ( - {0.04}_{4} * External {dysuria}_{4} ) + ( {0.65}_{6} * Candida Antigen {Positive}_{6} ) = 0.95 \qquad ( iii )$$

Based on the above, the probability of candidiasis is 95%.

In this exemplary embodiment, the same input data is shown to an expert 450 who determines the validity of the score. Input data 412, 422, 424, 432, output data 400 and expert opinion 450 are then fed to the machine-expert system 440 where decisions on adjusting input parameter probabilities are made and assigned to parameters. In this exemplary embodiment, new parameter probabilities are re-fed to the algorithm and the cycle is repeated until the system is concurrent or aligned with expert opinion 450. Unknown parameters such as X₂ non-structured 414 history information or as X₅ user provided symptom 426 symptom information may also be optionally added to the cycle and their effect on the diagnostic accuracy is extrapolated. Subsequently, probability is issued and assigned to said parameters. The parameter is then changed into calculated category and is re-assessed in future cycles. New tests parameters lacking pre-determined probabilities such as X₇ discharge photo analysis 434 and X₈ metabolomic spectrometry 436 may also be added to the cycle, compared, and may replace parameters with known probabilities such as X₆ candida antigen 432. This method may advantageously replace current diagnostic technologies as the method is more cost-effective and is supported by faster technologies.

In some embodiments, expert opinion 450 can be replaced by other methods of objective analysis of the diagnosis. In some embodiments, X₇ discharge photo analysis 434 parameter with various known symptoms 420 or history 410 categories parameters can be evaluated against DNA candida test 452 and the like. Additionally, sample photo analysis learning may be performed prior to execution in said machine expert system by learning the correct diagnosis from images of vaginal microscopy confirmed by experts to be strongly associated with said vulvovaginal conditions, using deep learning algorithms such as Convolutional Neural Networks (CNN), image restoration, linear filtering, pixelation, independent component analysis, template matching, Image Generation Techniques (GAN) and the like.

Therefore, in some embodiments, there is provided a method for diagnosing a vaginal disorder in a subject, said method comprises:
collecting primary data obtained from a device for diagnosing the vaginal disorder;
storing the primary data in a database;
sending the primary data to a processor, said processor is configured to run a plurality of modules comprising at least a diagnosis module and a treatment module and to process the primary data; and
determining the likelihood of the subject diagnosed with the vaginal disorder based on the data processed and if the subject is diagnosed with the vaginal disorder, recommending one or more treatment options.

Further, in some embodiments, there is also provided a system for diagnosing a vaginal disorder in a subject, said system comprises:
a device for analyzing a sample obtained from the subject;
a database for storing primary data, said primary data comprising a test result from said device;
a processor, configured to process the primary data and to run a plurality of modules comprising at least a diagnosis module and a treatment module, for providing the likelihood of the vaginal disorder in the subject based on the data processed.

It should be evident that this system will work in tandem with big data systems to find the most accurate, safe and cheap parameters for diagnosis for each condition causing vaginitis and other condition causing vulvovaginitis. The system and method disclosed herein may also contribute to current diagnosis of vulvar conditions using image analysis and patient driven data, urinary tract infection when using home urine stick, Tonsilitis when using rapid strep test and the like.

It should be appreciated that the above-described methods, systems and apparatus may be varied in many ways, including omitting, or adding elements or steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by a person skilled in the art that the present invention is not limited to what has been particularly shown and described hereinabove. Rather the scope of the present invention is defined by the claims, which follow.

1. A method for diagnosing a vaginal disorder in a subject, comprising:
collecting primary data obtained from a device for diagnosing the vaginal disorder;
storing the primary data in a database;
sending the primary data to a processor, said processor is configured to run a plurality of modules comprising an at least one diagnosis module and a treatment module and to process the primary data; and
determining a likelihood of the subject diagnosed with the vaginal disorder based on the data processed and if the subject is diagnosed with the vaginal disorder, recommending one or more treatment options.

2. The method of claim 1, further comprising collecting secondary data, said secondary data comprising structured data and non-structured data and sending the secondary data to the processor for processing said secondary data.

3. The method of claim 1, wherein diagnosing the vaginal disorder comprising diagnosing candidiasis, bacterial vaginosis and trichomoniasis or vaginitis.

4. The method of claim 1, wherein diagnosing the vaginal disorder comprising diagnosing vulvar conditions.

5. The method of claim 1, wherein the processor further comprising a computing element and a machine learning module, and wherein said computing element comprising the at least one diagnosis module and one treatment module.

6. The method of claim 5, wherein determining the likelihood of the subject diagnosed with the vaginal disorder is based on a probability score generated by the computing element.

7. The method of claim 1, wherein said primary data comprising pH, result of candida antigen test, result of sialidase activity test, result of DNA or RNA test, one or more images obtained from the subject and combination thereof.

8. The method of claim 7, wherein further comprising collecting secondary data, said secondary data comprising asking the subject is experiencing at least one of the following conditions: external vulvar pain, itching sensation and external vulvar discomfort.

9. The method of claim 7, wherein if the result of sialidase activity test is positive, the subject is experiencing at least one of the following conditions: fishy smell and increased or abnormal discharge.

10. The method of claim 7, wherein if the pH is greater than 4.5, the subject is recommended to undergo a trichomonas antigen test.

11. The method of claim 10, wherein if the result of the trichomonas antigen test is negative, the subject is asked if the subject is experiencing at least one of the following conditions: fishy smell and increased or abnormal discharge.

12. The method of claim 10, wherein if the result of the trichomonas antigen test is negative, the one or more images are analyzed by an image analysis module.

13. The method of claim 7, wherein if the result of the candida antigen test is negative and the pH is less than 4.5, the one or more images are analyzed by an image analysis module to confirm presence of Candida organism.

14. A system for diagnosing a vaginal disorder in a subject, comprising:
a device for analyzing a sample obtained from the subject;
a database for storing primary data, said primary data comprising a test result from said device; and
a processor, configured to process the primary data and to run a plurality of modules comprising at least a diagnosis module and a treatment module, for providing a likelihood of the vaginal disorder in the subject based on the data processed.

15. The system of claim 14, wherein said device comprising a collection apparatus for collecting and sampling vaginal discharge or fluid and a sensor for processing the sampling vaginal discharge, said sensor comprising at least two lateral flow assays (LFAs).

16. The system of claim 14, wherein said database further comprising secondary data, said secondary data comprising structured data and non-structured data.

17. The system of claim 14, wherein diagnosing the vaginal disorder comprising diagnosing vulvar conditions, candidiasis, bacterial vaginosis and trichomoniasis or vaginitis.

18. The system of claim 14, wherein the processor further comprising a computing element and a machine learning module, said computing element comprising a diagnosis module and a treatment module.

19. The system of claim 18, wherein the likelihood of the subject diagnosed with the vaginal disorder is based on a probability score generated by the computing element.

20. The system of claim 14, wherein said primary data comprising pH, result of candida antigen test, result of sialidase activity test, result of DNA or RNA test, one or more images obtained from the subject and combination thereof.

21. The system of claim 14, further comprising an amine sensor.
